SEQUENCE LISTING (45 sequences)

SEQ ID NO: 1 - DNA, 777 bp, artificial
  6cdtA DNA
gcccagccggccatggccgaggttcagctgcagcagtcaggacctggcctggtggcgccctcacagagcctgtccatcacttgcactgtctcagggttttctttaaccagttatggagtacactgggttcgccagcctccaggaaagggtctggagggactgggagtaatatgggctggtggaagcacaaattataattcggctctcatgtccagactgagcatcagcaaagacaactccaaagccaagttttcttaaaaatgaacagtctgcaaattgatgacacagccatgtattactgtgccagaagtcctacctccactatggatacgccctatgctatggactactggggtcaaggaacctcagtcaccgtcggcccttcaggcccatccggaggttcaggcggaggtggcggatcgcggtccggaacggatattttgatgacccaaactccactctccctgcctgtcagtcttggagatcaagcctccatgtcttgcagatctagtcagagccttgttcacaataatggagacacctatctgcactggtacctgcagaagccaggccagtctccaaagctcctgctccacaaagtttccaaccgcctttctggggtcccagacaggttcagtggcagtggatcagggacagatttcacactcaagatcagcagagtggagactgaggatctgggagtttatttctgctctcaaagtacacatgttccgtggacgttcggtggaggcaccaagctggaaataaaacgtcgga

SEQ ID NO: 2 - protein, 259 aa, artificial
  6cdtA PROTEIN
AQPAMAEVQLQQSGPGLVAPSQSLSITCTVSGFSLTSYGVHWVRQPPGKGLEGLGVIWAGGSTNYNSALMSRLSISKDNSKSQVFLKMNSLQIDDTAMYYCARSPTSTMDTPYAMDYWGQGTSVTVGPSGPSGGSGGGGGSRSGTDILMTQTPLSLPVSLGDQASMSCRSSQSLVHNNGDTYLHWYLQKPGQSPKLLLHKVSNRLSGVPDRFSGSGSGTDFTLKISRVETEDLGVYFCSQSTHVPWTFGGGTKLEIKRR

SEQ ID NO: 3 - DNA, 754 bp, artificial
  10cdtB DNA
gcccagccggccatggccgaggtccagctgcagcagtctgggggaggcttagtgaaacctggagggtccctgaaactctcctgtgcagcctctggattccctttcagtagctatgccatgtcttgggttcgccagactccggagaagaggctggagtgggtcgcaaccattactagtggtggttcttacacctactatccagacagtgtaaaggggcgattcaccatctccagagacaaagccaagaacaccctgtacctgcaaatgggcacactgaggtctgaggacacggccatgtattactgtgctagactccctgggagcaactatgctatggactactggggtcagggaacctcagtcaccgtcggcccttcaggcccatccggaggttcaggcggaggtggcggatcgcggtccggaacggaaaatgtgctcacccagtctccagccaccctatctgtgactccaggagatagcgtcagtctttcctgcagggccagcaaaagtattagcaacaatctacactggtatcaacaaaaatcacatgagtctccaagacttctcatcaagtatgcttctcagtccatttctgggatcccctccaggttcagtggcagtggatcagggacagatttcactcttagtatcaacagtgtggagaccgaagattttggaatgtatttctgtcaacagagtaacagctggccttacacgttcggaggggggaccaagctggaaataaaacgtcgga

SEQ ID NO: 4 - protein, 251 aa, artificial
  10cdtB PROTEIN
AQPAMAEVQLQQSGGGLVKPGGSLKLSCAASGFPFSSYAMSWVRQTPEKRLEWVATITSGGSYTYYPDSVKGRFTISRDKAKNTLYLQMGTLRSEDTAMYYCARLPGSNYAMDYWGQGTSVTVGPSGPSGGSGGGGGSRSGTENVLTQSPATLSVTPGDSVSLSCRASKSISNNLHWYQQKSHESPRLLIKYASQSISGIPSRFSGSGSGTDFTLSINSVETEDFGMYFCQQSNSWPYTFGGGTKLEIKRR

SEQ ID NO: 5 - DNA, 23 bp, artificial
  CH (heavy chain)
taagccctttgacacaggcatcc

SEQ ID NO: 6 - DNA, 18 bp, artificial
  CK (kappa light chain
cgttcactgccatcaatc

SEQ ID NO: 7 - DNA, 21 bp, artificial
  CL (lambda light chain)
ggaaggtggaaacagctggtg

SEQ ID NO: 8 - DNA, 53 bp, artificial
  VH1
ggaaccctttggcccagccggccatggccgcaggtctcagctgctcagcagtc

SEQ ID NO: 9 - DNA, 50 bp, artificial
  VH2
ggaaccctttggcccagccggccatggcccaggttcacctgcagcaagtc

SEQ ID NO: 10 - DNA, 50 bp, artificial
  VH3
ggaaccctttggcccagccggccatggcccaggtagcagctgaaggagtc

SEQ ID NO: 11 - DNA, 52 bp, artificial
  VH4
ggaaccctttggcccagccggccatggcccaggtccaactagccagcaagcc

SEQ ID NO: 12 - DNA, 50 bp, artificial
  VH5
ggaaccctttggcccagccggccatggcccagatccagttggtagcagtc

SEQ ID NO: 13 - DNA, 51 bp, artificial
  VH6
ggaaccctttggcccagccggccatggcccaggtgcagctgaaggcagctc

SEQ ID NO: 14 - DNA, 51 bp, artificial; identical to SEQ ID NO: 13
  VH7

SEQ ID NO: 15 - DNA, 51 bp, artificial
  VH8
ggaaccctttggcccagccggccatggccgaagtgaaaggcttgaggagtc

SEQ ID NO: 16 - DNA, 53 bp, artificial
  VH9
ggaaccctttggcccagccggccatggccgagtgtgcagcagcttcaggagtc

SEQ ID NO: 17 - DNA, 51 bp, artificial
  VH10
ggaaccctttggcccagccggccatggccgaggtgaagcgctggtggaatc

SEQ ID NO: 18 - DNA, 51 bp, artificial
  VH11
ggaaccctttggcccagccggccatggccgaggtgaagctgagtggaagtc

SEQ ID NO: 19 - DNA, 51 bp, artificial
  VH12
ggaaccctttggcccagccggccatggccgaaggtgaagctgagtggagtc

SEQ ID NO: 20 - DNA, 49 bp, artificial
  VH13
ggaaccctttggcccagccggccatggccgaagtgcagctgttggagac

SEQ ID NO: 21 - DNA, 50 bp, artificial
  VH14
ggaaccctttggcccagccggccatggccgaaggtgaagcttctcgcagt

SEQ ID NO: 22 - DNA, 49 bp, artificial
  VH15
ggaaccctttggcccagccggccatggcccaaggttactctgaaagagt

SEQ ID NO: 23 - DNA, 39 bp, artificial
  JH1.1
tccggatgggcctgaagggccgacggtgaccgtggtccc

SEQ ID NO: 24 - DNA, 39 bp, artificial
  JH2.1
tccggatgggcctgaagggccgactgtgagagtggtgcc

SEQ ID NO: 25 - DNA, 39 bp, artificial
  JH3.1
tccggatgggcctgaagggccgacagtgaccagagtccc

SEQ ID NO: 26 - DNA, 39 bp, artificial
  JH4.1
tccggatgggcctgaagggccgacggtgactgaggttcc

SEQ ID NO: 27 - DNA, 42 bp, artificial
  VK1.1
ggatcgcggtccggaacggatattgtgatgacgctcagagtc

SEQ ID NO: 28 - DNA, 41 bp, artificial
  VK2.1
ggatcgcggtccggaacggatagttgttgatgacccaagac

SEQ ID NO: 29 - DNA, 38 bp, artificial
  VK3.1
ggatcgcggtccggaacggaaaatgtgctcacccagtc

SEQ ID NO: 30 - DNA, 39 bp, artificial
  VK4.1
ggatcgcggtccggaacggactattgtgatgacacagtc

SEQ ID NO: 31 - DNA, 38 bp, artificial
  VK5.1
ggatcgcggtccggaacggacatccagatgacacagac

SEQ ID NO: 32 - DNA, 42 bp, artificial
  VK6.1
ggatcgcggtccggaacggactattgtgctgcacctcaagtc

SEQ ID NO: 33 - DNA, 40 bp, artificial
  VK7.1
ggatcgcggtccggaacggacatccagatgacctcaagtc

SEQ ID NO: 34 - DNA, 38 bp, artificial
  VK8.1
ggatcgcggtccggaacgcaaattgttctcacccagtc

SEQ ID NO: 35 - DNA, 33 bp, artificial
  JK1/2.1
cgcgccggtccgacgtttgtatttccagcttgg

SEQ ID NO: 36 - DNA, 32 bp, artificial
  JK4.1
cgcgccggtccgacgttttatttccaactttg

SEQ ID NO: 37 - DNA, 41 bp, artificial
  JK5.1
cgcgccggtccgacgtttcagctctttcagctccagcttgg

SEQ ID NO: 38 - DNA, 36 bp, artificial
  VL1.1
ggatcgcggtccggaacgcaggctgttgtgactcag

SEQ ID NO: 39 - DNA, 32 bp, artificial
  JL1
cgcgccggtccgacctaggacagtcagtttgg

SEQ ID NO: 40 - DNA, 32 bp, artificial
  JL2/3
cgcgccggtccgacctaggacagtgaccttgg

SEQ ID NO: 41 - DNA, 17 bp, artificial
  Heavy chain forward primer JH_F
ctttggcccagccggcc

SEQ ID NO: 42 - DNA, 39 bp, artificial
  Heavy chain reverse primerJH_R
acctccgcctgaacctccggaaggacctgaagggccgac

SEQ ID NO: 43 - DNA, 39 bp, artificial
  Light chain forward primer (lamda and kappa)JL_F
tccggaggttcaggcggaggtggctcggggtccggaacg

SEQ ID NO: 44 - DNA, 15 bp, artificial
  Lambda light chain reverse primerJLL_R
cgcgccggtccgacc

SEQ ID NO: 45 - DNA, 15 bp, artificial
  Kappa light chain reverse primerJLK_R
cgcgccggtccgacg

(19) United States
(12) Patent Application Publication
(10) Pub. No.: US 2011/0150907 A1
(43) Pub. Date: Jun. 23, 2011

(54) METHODS AND MATERIALS FOR GASTROINTESTINAL DELIVERY OF PATHOGEN/TOXIN BINDING AGENTS

(75) Inventors: Jos Seegers, Leiden (NL); Stephen F. Carroll, Walnut Creek, CA (US)

(73) Assignee: FARALLONE HOLDINGS BV, Amsterdam (NL)

(21) Appl. No.: 12/936,108

(22) PCT Filed: Apr. 3, 2009
(86) PCT No.: PCT/US09/39491

§ 371 Date: Mar. 14, 2011

Related U.S. Application Data
(60) Provisional application No. 61/042,541, filed on Apr. 4, 2008.

(51) Int. Cl. A61K 39/385; A61K 35/74; C12N 7/00; C12N 1/20; C12N 1/21; A61P 31/12; A61P 31/04; C07K 16/00

(52) U.S. Cl. 424/178.1; 424/93.2; 435/235.1; 435/252.1; 435/252.3; 435/252.7; 435/252.8; 530/387.1

(57) ABSTRACT

The present disclosure relates generally to recombinant bacteria (e.g., Lactobacillus) that express one or more binding peptides, antibodies and/or antibody binding fragments on their surface that are specific for one or more pathogens and/or toxins, including toxins from pathogens. The recombinant bacteria may be used for binding, removing and/or neutralizing one or more pathogens and/or toxins, including toxins from pathogens in a gastrointestinal tract.

### FIELD

The present disclosure relates generally to recombinant bacteria (e.g., Lactobacillus) that express one or more binding peptides, antibodies and/or binding fragments thereof on their surface that are specific for one or more pathogens and/or toxins, including toxins from pathogens. The recombinant bacteria may be used for binding, removing and/or neutralizing one or more pathogens and/or toxins, including toxins from pathogens in a gastrointestinal tract. More specifically, the disclosure relates generally to the use of these recombinant bacteria as a therapeutic or prophylactic agent for the prevention and/or treatment of gastrointestinal diseases, including, for example, C. difficile associated diarrhea (CDAD).

### BACKGROUND

The human gastrointestinal tract is a well balanced complex ecosystem of microbes that forms a natural barrier against many enteropathogens. The delicate balance of this ecosystem can be upset by antimicrobial treatments, such as antibiotics, and lead to the establishment of pathogens and toxins. For example, Clostridium difficile, a well known enteropathogen, is a Gram-positive spore forming bacterium that is often part of this ecosystem. In healthy individuals this bacterium is kept to low numbers by the microbes that are comprised in the microflora of the gastrointestinal tract. However, when the balance is disturbed, C. difficile may flourish and lead to nosocomial gastrointestinal diseases (e.g., mild diarrhea, fatal pseudomembranous colitis and C. difficile associated diarrhea (CDAD)) that are predominantly caused by released toxin A and toxin B. Major groups at risk of C. difficile infection are the elderly and immune compromised patients with infections resulting in prolonged hospitalization. C. difficile infection is diagnosed in over 350,000 patients on an annual basis in the US alone with costs to the health care system calculated to be in excess of US $1 billion annually. Currently, the most common way of treating CDAD is an antibiotic treatment with either metronidazole or vancomycin. However, these treatments are often inefficient and associated with undesired effects.

### SUMMARY

The present disclosure relates generally to recombinant bacteria (e.g., Lactobacillus) that comprise one or more binding peptides, antibodies and/or binding fragments thereof anchored to their surface which are specific for one or more pathogens and/or toxins, including toxins from pathogens (e.g., C. difficile cells and/or C. difficile toxins). Notably, the bacteria can be used as a treatment and/or prophylactic for a gastrointestinal disease (e.g., mild diarrhea, fatal pseudomembranous colitis or C. difficile associated diarrhea (CDAD)).

The present disclosure also relates generally to methods for treating a gastrointestinal disease in a subject in need thereof by administering to the subject one or more recombinant bacterium each comprising one or more binding agents, including, for example, binding peptides, antibodies or binding fragments thereof anchored to its surface and specific for one or more pathogens and/or toxins, including toxins from pathogens.

The present disclosure also provides methods for binding, removing and/or neutralizing one or more pathogens and/or toxins, including toxins from pathogens (e.g., C. difficile cells and/or C. difficile toxins) in a gastrointestinal tract by administering to a subject in need thereof one or more recombinant bacteria (e.g., Lactobacillus) that each comprises one or more binding peptides, antibodies and/or binding fragments thereof anchored to its surface which are specific for one or more pathogens and/or toxins, including toxins from pathogens.

The present disclosure also provides compositions for treating a gastrointestinal disease that comprise one or more recombinant bacterium comprising one or more binding peptides, antibodies or binding fragments thereof anchored to their surface and specific for one or more pathogens and/or toxins, including toxins from pathogens.

In some embodiments, the bacterium is a Lactobacillus strain. In further embodiments, the Lactobacillus strain is selected from the group consisting of: L. casei, L. paracasei, L. zeae, L. reuteri, L. plantarum, L. acidophilus, L. gasseri and L. brevis.

In some embodiments, the pathogen is a bacterium commonly found in the gastrointestinal tract. In some embodiments, the bacterium is selected from the group consisting of: enterotoxicogenic Escherichia coli, Campylobacter jejuni, Cryptosporidium spp., Giardia lamblia, Yersinia enterocolitica, Helicobacter pylori, all Clostridium spp. and Vibrio cholera. In some embodiments, the bacterium is C. difficile.

In some embodiments, the pathogen is a bacterium that is ingested, for example, from air, food and/or water. In further embodiments, the bacterium is selected from the group consisting of Salmonella, Shigella and Listeria spp.

In some embodiments the pathogen is a virus. In further embodiments, the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

In some embodiments, the antibodies or binding fragments thereof are anchored to the bacterium through a sortase dependent anchor sequence, a transmembrane anchor, a lipid anchor or an AcmA-like anchor. In some embodiments, the antibodies or binding fragments thereof are anchored to the bacterium by integration into a surface layer protein.

In some embodiments, the antibody is a single chain antibody. In other embodiments, the antibody binding fragment is selected from the group consisting of: Fab, Fab′, Fab′-SH, Fv, scFv, F(ab)₂, Vhh, nanobody and diabody.

In some embodiments, the antibody or fragments thereof are specific for Toxin A, Toxin B or surface antigens on C. difficile cells.

In some embodiments, the subject has mild diarrhea, fatal pseudomembranous colitis or C. difficile associated diarrhea (CDAD).

In some embodiments, the methods for treating include co-administering the recombinant bacteria with one or more agents such as antibiotic and/or antiviral agents (e.g., vancomycin, metronidazole).

### BRIEF DESCRIPTION OF THE FIGURES

FIG. 1: Graphic representation of a Lactobacillus single chain antibody cloning and expression vector. RepA and repC are replication proteins involved in plasmid replication and copy control. FG is a food grade selection marker for plasmid maintenance. Ss slpA indicates the position of the secretion signal. The position of the secretion signal (ss slpA), the E-tag sequence and the anchor sequence are indicated. Tcbh indicates the position of a Rho-independent transcriptional terminator sequence.

FIG. 2: Western blot analysis of expression of anti Toxin A (6cdtA) and anti Toxin B (10cdtB) scFv at the cell surface of Lactobacillus paracasei. WM is molecular weight marker; (−) is Lactobacillus paracasei, not expressing a scFv. Cells were grown in LCM to OD₆₀₀=0.7-1.0. Cells were harvested and washed with PBS. After resuspending the cells in PBS they were disrupted by sonication. The cell suspension was complemented with sample buffer, boiled for 10 minutes and loaded onto a polyacrylamide gel. Following gel electrophoresis proteins were transferred onto nitrocellulose membranes and detected with anti E-tag antibodies.

FIG. 3: Flow cytrometric analysis showing exposition of anti Clostridium difficile toxin on the bacterial cell surface. Cells, expressing anti C. difficile toxin scFv were grown in LCM to OD₆₀₀=0.7-1.0. Cells were washed with PBS and resuspended in PBS with mouse monoclonal anti-E-tag IgG antibodies. In a second step cells were incubated in the presence of FITC-labelled anti mouse IgG antibodies. Analysis was done in a FACScalibur flow cytometer (Beckton Dickinson, San Jose, Calif.). As a control a Lactobacillus strain was taken, carrying a plasmid without functional insert. Panel A is a histogram plot of the fluorescence of the wild-type strain (blue line) and the scFv expressing strain (green line). Panel B shows density plots of the wild type strain (pSLP111.1) and the scFv expressing strain.

FIG. 4: Immunofluorescence microscopy showing surface expression of the scFv in Lactobacillus. Left panel shows a normal light image of the bacteria, the right panel is a fluorescent image of the same bacteria as a result of FITC-labelled antibodies, bound to the cell surface of the bacteria.

FIG. 5: ELISA showing binding of 6cdtA and 10cdtB to Toxin A and B, respectively. 1=blank measurement, without scFv, 2=culture supernatant containing scFv of corresponding toxin, 3=purified scFv of corresponding toxin, 4=purified scFv of other toxin.

FIG. 6: Serial dilution ELISA showing binding of purified 10cdtB to Toxin B, 10cdtB supernatant to Toxin B or negative control.

FIG. 7: Graphical representation showing protection from diarrhea in animals infected with C. difficile and treated orally with Lactobacillus expressing single chain antibodies to Toxin A and Toxin B.

FIG. 8: Kaplan-Meier analysis showing survival of animals infected with C. difficile and treated orally with Lactobacillus control, Lactobacillus expressing surface bound single chain antibodies to Toxin A, Lactobacillus expressing single chain antibodies to Toxin B, or Lactobacillus expressing single chain antibodies to Toxin A and Toxin B.

### DETAILED DESCRIPTION

The present disclosure provides recombinant bacteria (e.g., Lactobacillus) that comprise one or more binding peptides, antibodies and/or binding fragments thereof anchored to their surface which are specific for one or more pathogens and/or toxins, including toxins from pathogens (e.g., C. difficile cells and/or C. difficile toxins). Surprisingly, the modified bacteria are capable of binding to a bacterial and/or viral pathogen and removing the pathogen from the gastrointestinal tract of an animal (e.g. a human). Accordingly, these modified bacteria can be used as a treatment and/or prophylactic for a gastrointestinal disease (e.g., mild diarrhea, fatal pseudomembranous colitis or C. difficile associated diarrhea (CDAD)).

The present disclosure also provides methods for treating a gastrointestinal disease in a subject in need thereof by administering to the subject one or more recombinant Lactobacillus comprising one or more binding agents, including, for example, binding peptides, antibodies and/or binding fragments thereof anchored to its surface and specific for one or more bacterial pathogens, viral pathogens and/or toxins, including toxins from pathogens.

The present disclosure also provides methods for binding one or more pathogens and/or toxins, including toxins from pathogens (e.g., C. difficile cells and/or C. difficile toxins) in a gastrointestinal tract by administering to a subject in need thereof one or more recombinant Lactobacillus that comprises one or more binding peptides, antibodies and/or binding fragments thereof anchored to its surface which are specific for one or more pathogens and/or toxins, including toxins from pathogens.

The present disclosure also provides methods for removing one or more pathogens and/or toxins, including toxins from pathogens (e.g., C. difficile cells and/or C. difficile toxins) in a gastrointestinal tract by administering to a subject in need thereof one or more recombinant Lactobacillus that comprises one or more binding peptides, antibodies and/or binding fragments thereof anchored to its surface which are specific for one or more pathogens and/or toxins, including toxins from pathogens.

The present disclosure also provides methods for neutralizing one or more pathogens and/or toxins, including toxins from pathogens (e.g., C. difficile cells and/or C. difficile toxins) in a gastrointestinal tract by administering to a subject in need thereof one or more recombinant Lactobacillus that comprises one or more binding peptides, antibodies and/or binding fragments thereof anchored to its surface which are specific for one or more pathogens and/or toxins, including toxins from pathogens.

The present disclosure also provides compositions for treating a gastrointestinal disease that comprise one or more recombinant Lactobacillus comprising one or more binding peptides, antibodies and/or antibody binding fragments anchored to its surface and specific for one or more bacterial pathogens, viral pathogens and/or toxins, including toxins from pathogens.

The present disclosure also provides compositions for treating a gastrointestinal disease that comprise one or more recombinant Lactobacillus comprising one or more binding peptides, antibodies and/or antibody binding fragments anchored to its surface and specific for one or more C. difficile toxins and/or C. difficile cells.

The present disclosure provides a recombinant Lactobacillus that comprises one or more binding peptide, antibodies and/or binding fragments thereof anchored to their surface which are specific for one or more bacterial pathogens, viral pathogens and/or toxins, including toxins from pathogens.

The present disclosure also provides a recombinant Lactobacillus that comprises one or more binding peptide, antibodies and/or binding fragments thereof anchored to their surface which are specific for one or more bacterial pathogens, viral pathogens and/or toxins, including toxins from pathogens.

The present disclosure also provides a recombinant Lactobacillus that comprises one or more binding peptide, antibodies and/or binding fragments thereof anchored to their surface which are specific for one or more C. difficile toxins and/or C. difficile cells.

In some embodiments, the Lactobacillus strain is selected from the group consisting of: L. casei, L. paracasei, L. zeae, L. reuteri, L. plantarum, L. acidophilus, L. gasseri and L. brevis.

In some embodiments, the pathogen is commonly found in the gastrointestinal tract. In further embodiments, the bacterium is selected from the group consisting of: enterotoxicogenic Escherichia coli, Campylobacter jejuni, Cryptosporidium spp., Giardia lamblia, Yersinia enterocolitica, Helicobacter pylori, all Clostridium spp. and Vibrio cholera. In some embodiments, the bacterium is C. difficile.

In some embodiments, the pathogen is a bacterium that is ingested (for example, from air, food and/or water). In further embodiments, the bacterium is selected from the group consisting of Salmonella, Shigella and Listeria spp.

In some embodiments the pathogen is a virus. In further embodiments, the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

In some embodiments, the binding peptides, antibodies or fragments thereof are anchored to the bacterium through a sortase dependent anchor sequence, a transmembrane anchor, a lipid anchor or an AcmA-like anchor. In some embodiments, the binding peptides, antibodies or fragments thereof are anchored to the bacterium by integration into a surface layer protein.

In some embodiments, the antibody is a single chain antibody. In other embodiments, the antibody binding fragment is selected from the group consisting of: Fab, Fab′, Fab′-SH, Fv, scFv, F(ab)₂, Vhh, nanobody and diabody.

In some embodiments, the subject has mild diarrhea, fatal pseudomembranous colitis or C. difficile associated diarrhea (CDAD).

In some embodiments, the methods for treating include co-administering the recombinant bacteria with one or more agents such as antibiotic and/or antiviral agents (e.g., vancomycin, metronidazole).

Although any methods and materials similar or equivalent to those described herein can be used in the practice or testing of the present disclosure, the preferred methods and materials are described.

### Binding Peptides, Antibodies and Binding Fragments Thereof

The recombinant bacterium (e.g., Lactobacillus) of the present disclosure may express one or more binding peptides, antibodies and/or binding fragments thereof anchored to its surface which are specific for one or more pathogens and/or toxins, including toxins from pathogens (e.g., C. difficile cells and/or C. difficile toxins).

In some embodiments, the binding peptide, antibody or binding fragment thereof is specific for Toxin A and/or Toxin B from C. difficile. Other pathogens and/or their toxins that may be targeted by one or more binding peptides, antibodies and/or fragments thereof include those described in Laohachai et al. (2003) Toxicon 42(7): 687-707), including, for example, (a) Vibrio cholerae (e.g., cholera toxin, E1 Tor hemolysin and accessory cholera enterotoxin); (b) Escherichia coli (e.g., heat stable enterotoxin, heat-labile enterotoxin and colicins); (c) Shigella dysenteriae (e.g., shiga-toxin and shiga-like toxin (e.g., a variant of shiga-toxin found in E. coli)); (d) Clostridium perfringens (e.g., C. perfringens enterotoxin, alpha-toxin, beta-toxin and theta-toxin); (e) Clostridium difficile (e.g., toxins A and B); (f) Staphylococcus aureus (e.g., alpha-haemolysin); (g) Bacillus cereus (e.g., cytotoxin K and haemolysin BL); and (h) Aeromonas hydrophila (e.g., aerolysin, heat labile cytotoxins and heat stable cytotoxins).

In some embodiments, the binding peptide, antibody or binding fragment thereof is specific for a pathogen of the gastrointestinal tract, such as, for example, enterotoxicogenic Escherichia coli, Campylobacter jejuni, Cryptosporidium spp., Giardia lamblia, Yersinia enterocolitica, Helicobacter pylori, all Clostridium spp. and Vibrio cholera. In yet another embodiment, the binding peptide or antibody is specific for a food borne pathogen, such as, for example, Salmonella, Shigella and Listeria spp. In yet another embodiment, the binding peptide or antibody is specific for a rotavirus, enteroviruses, adenoviruses, caliciviruses, reoviruses, coronaviruses, and Norwalk-type viruses, coxsackieviruses, poliovirus, hepatitis A virus.

Binding peptides contemplated by the present disclosure may include, but are not limited to repeat proteins such as, for example, darpins, ankyrin repeat proteins or leucine-rich repeat proteins (see, e.g., U.S. Patent Application Publication No. 2004/132028). In some embodiments, the binding peptide may be antibody-like (see, e.g., Hosse et al. (2006) Protein Science 15:14-27).

Various forms of an antibody are contemplated by the present disclosure. For example, the antibody may be an antibody fragment, such as a Fab, a Fab′, a Fab′-SH, a Fv, a scFv, a F(ab)₂, a Vhh, a nanobody and a diabody.

Many techniques have been developed for the production of antibody fragments. Traditionally, these fragments are derived via proteolytic digestion of intact antibodies (see, e.g., Morimoto et al., Journal of Biochemical and Biophysical Methods, 24:107-117 (1992); and Brennan et al., Science, 229:81 (1985)). However, these fragments can be produced directly by recombinant host cells. For example, the antibody fragments can be isolated from the antibody phage libraries. Alternatively, Fab′-SH fragments can be directly recovered from E. coli and chemically coupled to form F(ab′)₂ fragments (Carter et al., Bio/Technology, 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See WO 1993/16185; U.S. Pat. No. 5,571,894; and U.S. Pat. No. 5,587,458. The antibody fragment may also be a “linear antibody”, e.g., as described in U.S. Pat. No. 5,641,870, for example.

Single chain antibodies with specificity to a particular bacterial and/or viral pathogen can be obtained in a number of ways. For example, single chain antibodies to the bacterial and/or a viral pathogen, including, for example, C. difficile and/or its toxins, may be selected from a random library. This can be accomplished by phage display or any other technique that is commonly used for selection of high affinity molecules, such as ribosome display. This technique requires a redundancy of at least 10⁹, but preferable 10¹²-10¹⁴ to be successful. Positive binders are selected by panning against immobilized bacterial and/or viral pathogen. In another exemplary method, a mouse, rabbit or sheep is immunized with a bacterial and/or viral pathogen, including, for example, C. difficile and/or its inactivated toxins. RNA of immunized animals can be enriched for antibodies that are specific for a bacterial and/or viral pathogen. As a result, redundancy of the bank from which single chain antibodies need to be selected can be greatly reduced to 10⁵-10⁷, thereby increasing the chance of selecting positive binders. Because of the reduced size of the bank that is needed, positive binders can be selected through bacterial display, using a bacterium (e.g., Lactobacillus) as the expression host, in combination with magnetic beads, coated with the bacterial and/or viral pathogen (e.g., Toxin A or Toxin B or whole cells of killed C. difficile).

According to a different approach, antibody-variable domains with the desired binding specificities (antibody-antigen combining sites) to a bacterial and/or viral pathogen may be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for flexibility in adjusting the mutual proportions of the three polypeptide fragments in embodiments when unequal ratios of the three polypeptide chains used in the construction provide optimized yields. It is, however, possible to insert the coding sequences for the two or three polypeptide chains in one expression vector when the expression of at least two polypeptide chains in equal ratios results in high yields or when the ratios are of no particular significance.

### Vectors, Host Cells and Recombinant Methods

The present disclosure provides isolated nucleic acids encoding binding peptides, antibodies or binding fragments thereof specific for bacterial pathogens, viral pathogens and/or toxins, including toxins from pathogens, vectors and host cells comprising the nucleic acid, and recombinant techniques for the production and expression of the binding peptides, antibodies or binding fragments.

For recombinant production of a binding peptide, antibody or binding fragment thereof, the nucleic acid encoding it may be isolated and inserted into a replicable vector for further cloning (amplification of the DNA) or for expression. DNA encoding a binding peptide or antibody may be isolated and sequenced using conventional procedures (e.g., by using oligonucleotide probes that are capable of binding specifically to genes encoding heavy and light chains of an antibody). Vector components generally include, but are not limited to, one or more of the following: a signal sequence, an origin of replication, one or more marker genes, an enhancer element, a promoter, and a transcription-termination sequence. Binding peptides, antibodies or binding fragment thereof specific for a bacterial and/or viral pathogen may be expressed on the surface of a host cell (e.g. a recombinant bacterium).

### (i) Anchoring of Binding Peptide, Antibody or Fragment Thereof to Cell Surface

Binding peptides, antibodies or binding fragments thereof can be anchored to a cell surface. For example, binding peptides or antibodies may be anchored to the surface of a bacterium (e.g., a Lactobacillus species, such as L. casei, L. paracasei, L. zeae, L. reuteri and L. plantarum, L. acidophilus, L. gasseri, and L. brevis) through a sortase dependent anchor sequence or integrated into the surface layer of surface layer protein. Alternatively, binding peptides or antibodies may be attached to a cell surface through other methods, including but not restricted to the use of transmembrane anchors, lipid anchors or AcmA like anchors (see, for example, Leenhouts et al. (1999) Antonie van Leeuwenhoek 76: 367-376; and Deng et al. (2003) Clinical and Diagnostic Laboratory Technology 10(4): 587-595).

Anchoring of a binding peptide and/or antibody to a bacterial cell wall may be achieved by cloning of the binding peptide or antibody upstream and in frame with a sortase dependent anchor sequence. In a preferred embodiment, the anchor sequence is derived from the neutral protease PrtP. Optionally, the binding peptide or antibody may be joined in frame with a PrtP anchor and an E-tag sequence for easy detection of expression, using E-tag specific antibodies. Expression can be detected through western analysis or flow cytometry. Alternatively, flow cytometry conducted on intact cells can give direct evidence that the protein is expressed at the cell surface.

### (ii) Signal Sequence Component

Binding peptides, antibodies or binding fragments thereof specific for a bacterial and/or viral pathogen as described herein may be produced recombinantly not only directly, but also as a fusion polypeptide with a heterologous polypeptide, which is preferably a signal sequence or other polypeptide having a specific cleavage site at the N-terminus of the mature protein or polypeptide. A heterologous signal sequence preferably may be one that is recognized and processed (i.e., cleaved by a signal peptidase) by the host cell. For prokaryotic host cells that do not recognize and process the native binding peptide or antibody signal sequence, the signal sequence may be substituted by a prokaryotic signal sequence selected from the group of alkaline phosphatase, penicillinase, Ipp, or heat-stable enterotoxin II leaders. For yeast secretion a native signal sequence may be substituted by, e.g., a yeast invertase leader, a α-factor leader (including, for example, Saccharomyces and Kluyveromyces α-factor leaders), an acid-phosphatase leader, a C. albicans glucoamylase leader (see, e.g., WO 1990/13646).

(iii) Origin of Replication Component

Both expression and cloning vectors contain a nucleic acid sequence that enables the vector to replicate in one or more selected host cells. Generally, in cloning vectors this sequence may enable the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

### (iv) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply necessary or desired nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin. Alternatively a food grade selection marker may be used, including, for example, auxotrophic selection markers, such as lacF for lactose metabolism or bacterial lantibiotics such as lacticin 3147 from Lactococcus lactis subspecies Lactis DPC3147.

### (v) Promoter Component

Expression and cloning vectors usually contain a promoter that may be recognized by the host organism and may be operably linked to the humanized vWF antibody-encoding nucleic acid. Promoters suitable for use with prokaryotic hosts include a phoA promoter, β-lactamase and lactose promoter systems, alkaline phosphatase, a tryptophan (trp) promoter system, and hybrid promoters such as a tac promoter. However, other known bacterial promoters are suitable. Promoters for use in bacterial systems also may contain a Shine-Dalgarno (S.D.) sequence operably linked to the DNA encoding a binding peptide, antibody or binding fragment thereof specific for a bacterial and/or viral pathogen. In some embodiments, a constitutive promoter sequence may be used for expression of the binding peptide or antibody.

### (vi) Enhancer Element Component

Transcription of a DNA encoding a binding peptide, antibody or binding fragment thereof specific for a bacterial and/or viral pathogen may be increased by inserting an enhancer sequence into the vector. Useful enhancer sequences are now known from mammalian genes (globin, elastase, albumin, α-fetoprotein, and insulin). Typically, however, one may use an enhancer sequence from a eukaryotic cell virus are also useful. Examples include the SV40 enhancer on the late side of the replication origin (bp 100-270), the cytomegalovirus early-promoter enhancer, the polyoma enhancer on the late side of the replication origin, and adenovirus enhancers. Yaniv, Nature, 297:17-18 (1982) also describes enhancing elements for activation of eukaryotic promoters. An enhancer may be spliced into the vector at a position 5′ or 3′ to the humanized vWF antibody-encoding sequence, but is preferably located at a site 5′ from the promoter.

(vii) Transcription Termination Component

Expression vectors used in eukaryotic host cells (for example, yeast, fungi, insect, plant, animal, human, or nucleated cells from other multicellular organisms) may contain sequences necessary for the termination of transcription and for stabilizing the mRNA. Such sequences are commonly available from the 5′ end, occasionally 3′ end, of untranslated regions of eukaryotic or viral DNAs or cDNAs. These regions contain nucleotide segments transcribed as polyadenylated fragments in the untranslated portion of the mRNA encoding humanized vWF antibody. One useful transcription termination component is a bovine growth hormone polyadenylation region (see, e.g., WO 1994/11026 and expression vectors disclosed therein).

(viii) Secretion Signal

Secretion of a binding peptide or antibody to a cell surface may be effectuated by the use of a secretion signal. Genomic analysis of Lactobacilli has shown the presence of many surface anchored proteins. The sortase dependent secretion signals can be identified through the presence of a specific amino acid sequence at the C-terminal part of the protein. For SrtA this sequence may be LPXTG. Other suitable secretion signals of this class include a secretion signal from a Lactobacillus derived secretion signal, a secretion signal for alpha amylase, an aggregation promoting factor or a surface layer protein. Several other sortases have been identified and are contemplated for use with the present disclosure (see, e.g., Mazmanian et al. (2002) PNAS USA 99:2293-2298; and Barnett et al. (2004) J. Bacteriol. 186:5865-5875).

### (ix) Selection and Transformation of Host Cells

Suitable host cells for cloning or expressing the DNA in vectors include various prokaryote (e.g., bacteria, including for example, Lactobacillus), yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. E. coli cloning hosts include E. coli 294 (ATCC 31,446), E. coli B, E. coli X1776 (ATCC 31,537), and E. coli W3110 (ATCC 27,325).

Other host cells contemplated by the present disclosure include, but are not limited to Lactobacillus strains. For example, the Lactobacillus strain may be L. casei, L. paracasei, L. zeae, L. reuteri, L. plantarum, L. acidophilus, L. gasseri or L. brevis.

### (x) Culturing the Host Cells

Host cells, including but not limited to a bacterial cell (e.g., Lactobacillus), useful for producing a binding peptide, antibody or binding fragment thereof may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described, for example, in Ham et al., Meth. Enz. 58:44 (1979); Barnes et al., Anal. Biochem., 102:255 (1980); U.S. Pat. Nos. 4,767,704; 4,657,866; 4,927,762; 4,560,655; or 5,122,469; WO 1990/03430; WO 1987/00195; or U.S. Pat. Re. 30,985 may be used as culture media for the host cells. Any of these media may be supplemented as necessary with hormones and/or other growth factors (e.g., insulin, transferrin, or epidermal growth factor), salts (e.g., sodium chloride, calcium, magnesium, and phosphate), buffers (e.g., HEPES), nucleotides (e.g., adenosine and thymidine), antibiotics (e.g., GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. A variety of culture conditions, such as temperature, pH, maybe used with the host cell selected for expression.

### Methods of Treatment

Recombinant bacteria that comprise one or more binding peptides, antibodies and/or binding fragments thereof anchored to their surface which are specific for one or more bacterial pathogens, viral pathogens and/or toxins, including toxins from pathogens (e.g., C. difficile cells and/or C. difficile toxins) may be used to treat and/or prevent one or more gastrointestinal diseases/disorders in a subject in need thereof.

In some embodiments, one recombinant bacterial strain comprising one or more binding peptides, antibodies, or binding fragments thereof specific for one or more bacterial pathogens, viral pathogens and/or toxins (including toxins from pathogens) may be administered to a subject to treat a gastrointestinal disease or disorder. Alternatively, more that one (e.g., 2, 3, 4, 5, 6, 7, or 8) recombinant bacterial strains, each comprising one or more binding peptides, antibodies, or binding fragments thereof specific for the same or different bacterial pathogens, viral pathogens and/or toxins (including toxins from pathogens) may be administered to a subject to treat a gastrointestinal disease or disorder.

Gastrointestinal diseases and/or disorders are those caused by a bacterial pathogen, viral pathogen and/or toxin, including a toxin from a pathogen. In some embodiments, the gastrointestinal disease is caused by a bacterium commonly found in the gastrointestinal tract, including but not limited to, Escherichia coli, Campylobacter jejuni, Cryptosporidium spp., Giardia lamblia, Yersinia enterocolitica, Helicobacter pylori, all Clostridium spp., C. difficile and Vibrio cholera. In some embodiments, the gastrointestinal disease is caused by a bacterial pathogen that is ingested, for example, from consuming air, water and/or food. Exemplary bacteria include, but not limited to, Salmonella, Shigella and Listeria spp. In some embodiments gastrointestinal disease is caused by a virus, including, but not limited to rotavirus, enteroviruses, adenoviruses, caliciviruses, reoviruses, coronaviruses, Norwalk-type viruses, coxsackieviruses, poliovirus and hepatitis A virus.

Exemplary diseases or disorders that may be treated by the presently disclosed methods, include but are not limited to, mild diarrhea, fatal pseudomembranous colitis or C. difficile associated diarrhea (CDAD).

Compositions comprising the recombinant bacteria can be freeze dried and encapsulated in tablet form. In this way they can be stored at ambient temperature for at least one year without losing potency. Alternatively, the bacteria may be grown in liquid culture and stored at 4° C.

The recombinant bacteria may be administered to an animal, including for example, a human, in accordance with known methods. Treatment and/or methods of treating include prophylactic and/or therapeutic use of the recombinant bacteria alone or in combination with other agents such as antibiotics and/or antiviral agents. Such agents include, for example, vancomycin and metronidazole, OPT-80, Rifaximin (Xifaxan), Rifampin, Nitazoxanide, intravenous immunoglobulin G (IVIG), tolevamer potassium-sodium (GT267-004), Biological: GS-CDA1; Biological: MDX-1388 (systemic antibodies to TxA and TxB), GT160-246, MucoMilk product, as well as other prophylactic and/or therapeutic agents. For prophylactic and/or therapeutic use the bacteria that express one or more binding peptides, antibodies or binding fragments thereof are administered orally. Oral administration may be preformed in different formulations. Therapy can begin immediately following diagnosis or prior to exposure to prevent infection. The dosage may be at least twice a day with a minimal dosage size of 10¹⁰ bacteria. Treatment can be continued to one week after possible exposure or until the thread of exposure has ceased.

For the prevention or treatment of disease, the appropriate dosage of recombinant bacterium depends on the type of disease to be treated, the severity and course of the disease, whether the antibody may be administered for preventive or therapeutic purposes, previous therapy, the patient's clinical history and response to the recombinant bacterium, and the discretion of the attending physician. The recombinant bacterium may be suitably administered to the patient at one time or over a series of treatments. A preferred administration schedule may be at least twice a day with a minimal dosage size of 10⁴ to 10¹² bacteria (e.g., 10⁴, 10⁵, 10⁶, 10⁷, 10⁸, 10⁹, 10¹⁰, 10¹¹, or 10¹² bacteria). Treatment should be continued to one week after possible exposure or until the threat of exposure has ceased. However, other administration schedules are operable herein. Treatment (e.g., by feeding) preferably occurs one week prior to expected exposure to the pathogen and continues for one week after exposure or until the thread of infection has ceased. For repeated administrations over several days or longer, depending on the condition, the treatment may be sustained until a desired suppression of disease symptoms occurs.

Other therapeutic regimens may be combined with the administration of the recombinant bacteria, for example, other agents, including prophylactic or therapeutic agents, such as antibiotics and/or antivirals may be co-administered (e.g., before, with or after) the recombinant bacteria, including before, during or after infection. A combined administration includes co-administration, using separate formulations or a single pharmaceutical formulation, and consecutive administration in either order, wherein preferably there may be a time period while both (or all) active agents simultaneously exert their biological activities.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Expression and Functionality of Single Chain Antibodies, Directed Against Toxin A and B of C. Difficile, Produced by Lactobacillus

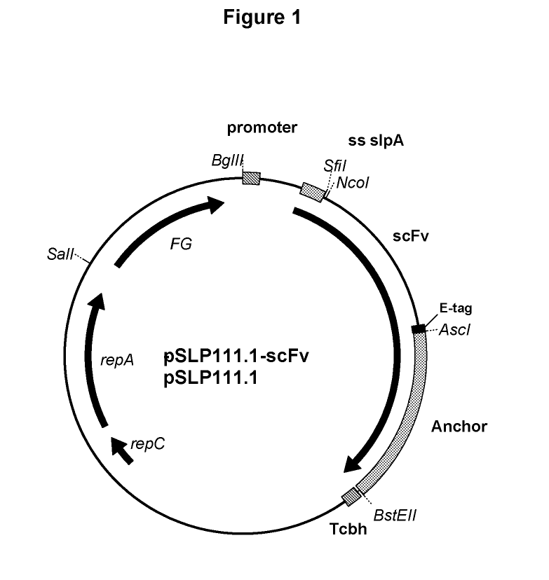

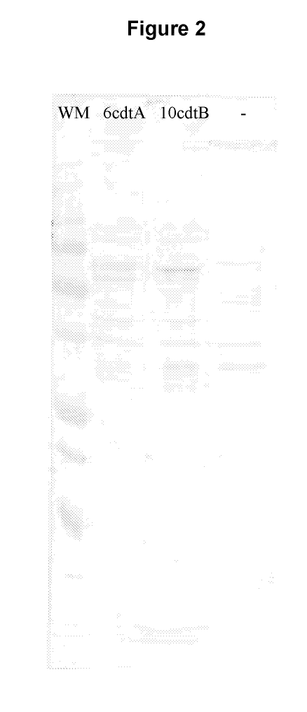

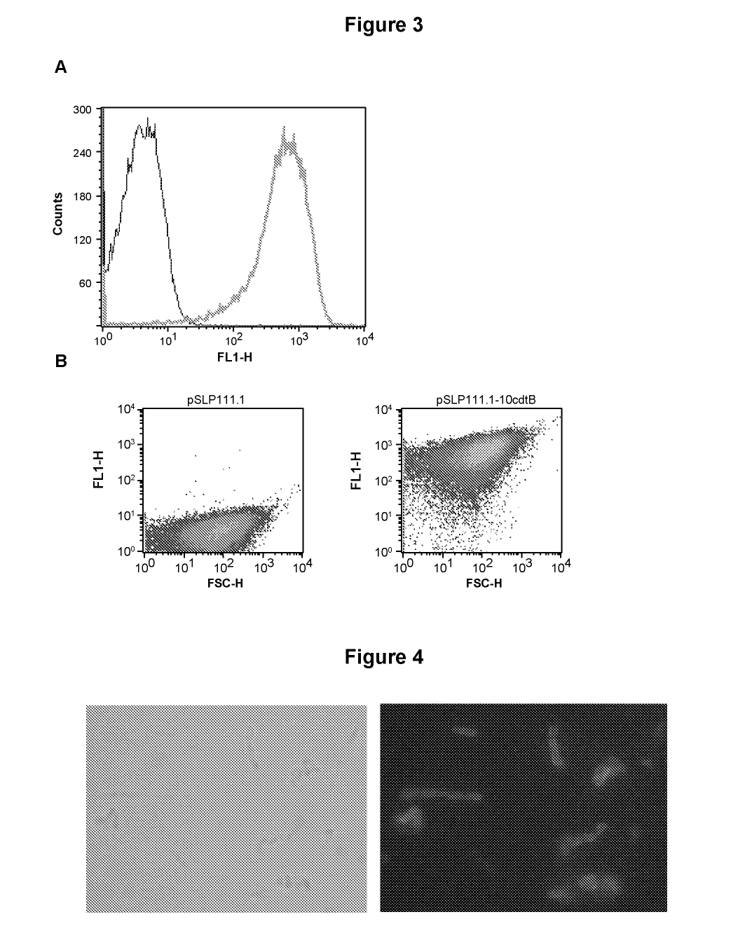

Single chain antibody fragments (scFv's) isolated from hybridoma's, expressing antibodies 6cdtA (SEQ ID NO: 2) and 10cdtB (SEQ ID NO: 4), specific for Toxin A and Toxin B, respectively, are cloned in frame with a Lactobacillus secretion signal at the N-terminal part of the protein and a sortase dependent anchor sequence at the C-terminal part of the protein (FIG. 1). The region between the promoter sequence and the slpA secretion signal sequence is a 190 base pair untranslated stability promoting leader sequence, derived from the original slpA gene region. This sequence stabilizes mRNA and leads to higher copy numbers and therefore increased expression levels. At the C-terminal part of the scFv an E-tag sequence was incorporated, allowing easy detection and one step purification of the antibody fragments. Expression of the protein was tested, using western analysis (FIG. 2). Confirmation that the expressed protein was actually present at the cell surface was obtained by flow cytometry and immunofluorescence microscopy (FIGS. 3 and 4).

### Example 2

### Binding of 6cdtA to Toxin A and 10cdtB to Toxin B

To determine if the single chain antibodies generated by Lactobacilli are capable of binding their respective toxins, the genes encoding the single chain antibodies were cloned in identical vectors as the pSLP111.1 vector in the absence of an anchor sequence. Single chain antibodies can than be purified in a one step E-tag affinity column purification and used for ELISA.

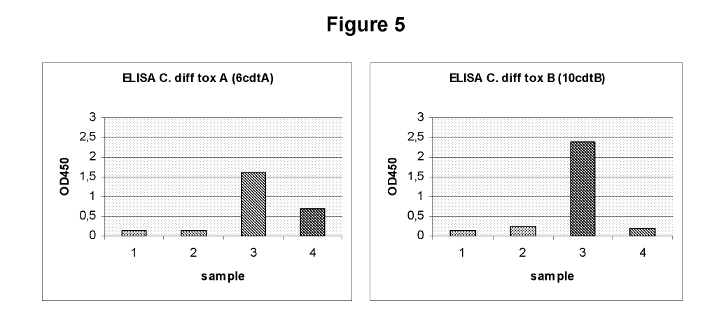

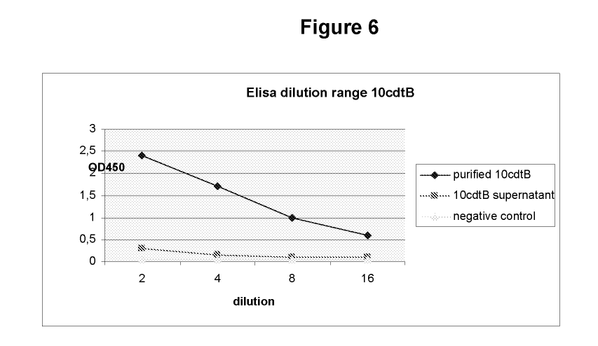

Briefly, plates were coated with either Toxin A or Toxin B at a concentration of 1 μg/ml. Blocking is performed with 2% skimmed milk in PBS. Plates are incubated with either purified scFv or pH adjusted supernatant, containing the anti toxin scFv. For toxin A coated plates the anti Toxin B scFv is used as a negative control, while for the anti Toxin B coated plates the anti Toxin A scFv is used as a negative control. The outcome of the ELISA is given in FIG. 5. These results show that 6cdtA binds to toxin A and that 10cdtB binds to toxin B. A positive signal was also observed for 10cdtB in Toxin A plates. This is due to the fact that the Toxin samples are not 100% pure and can still contain certain amounts of the other Toxin. Additionally binding of 10cdtB to toxin B was shown in a serial dilution ELISA as shown in FIG. 6.

### Example 3

### Selecting C. Difficile Specific Antibody Fragments from Mice, Immunized with Inactivated Whole Cell Bacteria

BALB/c mice are immunized with radiation killed whole cells of Clostridium difficile in combination with Freunds complete adjuvant. After six weeks serum is tested and mice are given a booster immunization. Two weeks after booster immunization serum is taken and tested for C. difficile antigen response. Mice are sacrificed and total RNA is isolated from their spleen. Using specific primer sets (see, e.g., Table 1) heavy and light chain fragments are amplified and joined via splicing by overlap extension. Following the first round of amplification all heavy chain fragments, lambda chain light fragments and kappa chain light fragments are mixed separately. These are further amplified with primers that are equipped with the proper restriction enzyme sites, which allows direct cloning into Lactobacillus expression vectors. For example, the amplified heavy chain fragments are further amplified with heavy chain forward primer JH_F (SEQ ID NO: 41) and heavy chain reverse primer JH_R (SEQ ID NO: 42), the amplified lambda light chains are amplified with light chain forward primer (lambda and kappa) JL_F (SEQ ID NO: 43) and lambda light chain reverse primer JLL_R (SEQ ID NO: 44), while the kappa light chains are further amplified with light chain forward primer (lambda and kappa) JL_F (SEQ ID NO: 43) and kappa light chain reverse primer JLK_R (SEQ ID NO 46). In addition, the 3′ heavy chain primers (reverse) and the 5′ light chain primers (forward) contain overlapping sequence that allows joining of heavy and light chain fragments by overlap extension. Through high efficiency electroporation the ligation mix is used to transform Lactobacillus paracasei. A total number of 10⁵ to 10⁷ transformants may be achieved. Positive binders are then selected from this bank by incubation with magnetic beads that are coated with radiation killed Clostridium difficile cells.

TABLE 1
Primers used for the generation of cDNA, amplification of
heavy and light chain and re-amplification of heavy and
light chain for combining heavy and light chain to one scFv

| | | |
|---|---|---|
| SEQ | | |
| ID NO: | Primer Name | Sequence |

Primers for the generation of cDNA

| | | |
|---|---|---|
| 5 | CH | TA(AG)CC(CT)TTGAC(AC)AGGCATCC |
| | (heavy chain) | |
| 6 | CK (kappa | CGTTCACTGCCATCAATC |
| | light chain | |
| 7 | CL (lambda | GGAAGGTGGAAACA(GCT)GGTG |
| | light chain) | |

Heavy chain forward primers

| | | |
|---|---|---|
| 8 | VH1 | GGAACCCTTTGGCCCAGCCGGCCATGGCC(G |
| | | C)AGGT(CT)CAGCT(GCT)CAGCAGTC |
| 9 | VH2 | GGAACCCTTTGGCCCAGCCGGCCATGGCCCA |
| | | GGTTCACCTGCAGCA(AG)TC |
| 10 | VH3 | GGAACCCTTTGGCCCAGCCGGCCATGGCCCA |
| | | GGT(AG)CAGCTGAAGGAGTC |
| 11 | VH4 | GGAACCCTTTGGCCCAGCCGGCCATGGCCCA |
| | | GGTCCAACT(AGC)CAGCA(AG)CC |
| 12 | VH5 | GGAACCCTTTGGCCCAGCCGGCCATGGCCCA |
| | | GATCCAGTTGGT(AGC)AGTC |
| 13 | VH6 | GGAACCCTTTGGCCCAGCCGGCCATGGCCCA |
| | | GGTGCAGCTGAAG(GC)A(GC)TC |
| 14 | VH7 | GGAACCCTTTGGCCCAGCCGGCCATGGCCCA |
| | | GGTGCAGCTGAAG(GC)A(GC)TC |
| 15 | VH8 | GGAACCCTTTGGCCCAGCCGGCCATGGCCGA |
| | | AGTGAA(AG)(GC)TTGAGGAGTC |
| 16 | VH9 | GGAACCCTTTGGCCCAGCCGGCCATGGCCGA |
| | | (GT)GT(GC)(AGC)AGCTTCAGGAGTC |
| 17 | VH10 | GGAACCCTTTGGCCCAGCCGGCCATGGCCGA |
| | | GGTGAA(GC)(GC)TGGTGGAATC |
| 18 | VH11 | GGAACCCTTTGGCCCAGCCGGCCATGGCCGA |
| | | GGTGAAGCTG(AG)TGGA(AG)TC |
| 19 | VH12 | GGAACCCTTTGGCCCAGCCGGCCATGGCCGA |
| | | (AG)GTGAAGCTG(AG)TGGAGTC |
| 20 | VH13 | GGAACCCTTTGGCCCAGCCGGCCATGGCCGA |
| | | AGTGCAGCTGTTGGAGAC |
| 21 | VH14 | GGAACCCTTTGGCCCAGCCGGCCATGGCCGA |
| | | (AG)GTGAAGCTTCTC(GC)AGT |
| 22 | VH15 | GGAACCCTTTGGCCCAGCCGGCCATGGCCCA |
| | | (AG)GTTACTCTGAAAGAGT |

Heavy chain reverse primers

| | | |
|---|---|---|
| 23 | JH1.1 | TCCGGATGGGCCTGAAGGGCCGACGGTGACC |
| | | GTGGTCCC |
| 24 | JH2.1 | TCCGGATGGGCCTGAAGGGCCGACTGTGAGA |
| | | GTGGTGCC |
| 25 | JH3.1 | TCCGGATGGGCCTGAAGGGCCGACAGTGACC |
| | | AGAGTCCC |
| 26 | JH4.1 | TCCGGATGGGCCTGAAGGGCCGACGGTGACT |
| | | GAGGTTCC |

Kappa light chain forward primer

| | | |
|---|---|---|
| 27 | VK1.1 | GGATCGCGGTCCGGAACGGATATTGTGATGA |
| | | C(GCT)CAG(AGT)C |
| 28 | VK2.1 | GGATCGCGGTCCGGAACGGAT(AG)TT(GT)TG |
| | | ATGACCCA(AG)AC |
| 29 | VK3.1 | GGATCGCGGTCCGGAACGGAAAATGTGCTCA |
| | | CCCAGTC |
| 30 | VK4.1 | GGATCGCGGTCCGGAACGGA(CT)ATTGTGAT |
| | | GACACAGTC |
| 31 | VK5.1 | GGATCGCGGTCCGGAACGGACATCCAGATGA |
| | | CACAGAC |
| 32 | VK6.1 | GGATCGCGGTCCGGAACGGA(CT)ATTGTGCT |
| | | (GC)AC(CT)CA(AG)TC |
| 33 | VK7.1 | GGATCGCGGTCCGGAACGGACATCCAGATGA |
| | | C(CT)CA(AG)TC |
| 34 | VK8.1 | GGATCGCGGTCCGGAACGCAAATTGTTCTCAC |
| | | CCAGTC |

Kappa light chain reverse primers

| | | |
|---|---|---|
| 35 | JK1/2.1 | CGCGCCGGTCCGACGTTT(GT)ATTTCCAGCTT |
| | | GG |
| 36 | JK4.1 | CGCGCCGGTCCGACGTTTTATTTCCAACTTTG |
| 37 | JK5.1 | CGCGCCGGTCCGACGTTTCAGCTCTTTCAGCT |
| | | CCAGCTTGG |

Lambda light chain forward primers

| | | |
|---|---|---|
| 38 | VL1.1 | GGATCGCGGTCCGGAACGCAGGCTGTTGTGA |
| | | CTCAG |

Lambda light chain reverse primers

| | | |
|---|---|---|
| 39 | JL1 | CGCGCCGGTCCGACCTAGGACAGTCAGTTTGG |
| 40 | JL2/3 | CGCGCCGGTCCGACCTAGGACAGTGACCTTGG |

Primers for re-amplification of heavy and light chains

| | | |
|---|---|---|
| 41 | Heavy chain | CTTTGGCCCAGCCGGCC |
| | forward | |
| | primer JH_F | |
| 42 | Heavy chain | ACCTCCGCCTGAACCTCCGGAAGGACCTGAA |
| | reverse | GGGCCGAC |
| | primer JH_R | |
| 43 | Light chain | TCCGGAGGTTCAGGCGGAGGTGGCTCGGGGT |
| | forward primer | CCGGAACG |
| | (lamda and | |
| | kappa)JL_F | |
| 44 | Lambda light | CGCGCCGGTCCGACC |
| | chain reverse | |
| | primer JLL_R | |
| 45 | Kappa light | CGCGCCGGTCCGACG |
| | chain reverse | |
| | primer JLK_R | |

### Example 4

### Protection Studies of Animals with C. Difficile Specific Single Chain Antibody Producing Lactobacilli

Efficacy of L. casei expressing anti Toxin A and Toxin B single chain fragment variable (scFv) for the treatment of C. difficile infection is determined in validated animals models. Hamsters are pretreated with L. paracasei expressing the anti toxin scFv and treatment is continued for one week following C. difficile infection.

Briefly, 6 to 7 week old hamsters are used for challenge studies. After hamsters have been assigned to treatment groups on the basis of mass, animals are fed a standard laboratory diet ad libitum. All animals are caged individually in isolator cages with disposable air filters to prevent cross contamination. Measures are taken to prevent secondary infections from occurring and animals are tested for C. difficile carriage by culturing the bacterium from faeces. Two days prior to infection, animals are treated with Lactobacillus by feeding animals 10⁴ to 10¹⁰ (e.g., 10¹⁰) CFU of Lactobacillus, two times per day. Animals are then split into one of five groups. One group is fed a Lactobacillus control strain that does not express any scFv, one group is fed a Lactobacillus strain that expresses surface bound 6cdtA scFv, one group is fed a Lactobacillus strain expressing surface bound 10cdtB scFv, one group is fed a mix of Lactobacillus strains expressing surface bound 6cdtA scFv or surface bound 10cdtB scFv and one group is not fed any Lactobacilli. On day three hamsters are given a two milligram dose of clindamycin-HCl orogastrically to predispose them to C. difficile infection. The animals are challenged with 10⁵ CFU four hours later. Lactobacillus strains are fed to the respective groups for one week. From the day after infection, hamsters are observed every two hours in a blinded fashion by three individual observers for seventy-two hours, and four times a day at regular intervals thereafter. Grading is conducted as follows: 0, normal; 1, loose faeces or wet perianum, activity close to normal; 2, reduced activity, still responding to stimuli, tender abdomen; 3, hunched, inactive, tender abdomen, loss of balance, ruffled fur. Hamsters are sacrificed at grade 3. Time of sacrifice or last time seen alive (whichever was earlier) is considered the endpoint.

To confirm C. difficile as the causative agent of disease, perianal swabs (no formed faeces due to diarrhoea) are taken in a random order from a representative number of symptomatic hamsters (n=5) and cultured anaerobically for four days on blood agar plates containing 50 μg/ml clindamycin. Caecum and colon samples are taken from a representative number of animals (one hamster per group) to confirm the typical epithelial damage seen in CDAD. Tissues are fixed in 10% formalin and stained with haematoxylin and eosin.

### Example 5

### Additional Protection Studies of Animals with C. Difficile Specific Single Chain Antibody Fragment Producing Lactobacilli

Efficacy of L. casei expressing anti Toxin A and Toxin B single chain variable fragments (scFvs) or other antibody fragments for the treatment of C. difficile infection may be determined in validated animal models.

Briefly, 6 to 7 week old hamsters are used for challenge studies. After hamsters are assigned to treatment groups on the basis of mass, animals are fed a standard laboratory diet ad libitum. All animals are caged individually in isolator cages with disposable air filters to prevent cross contamination. Measures are taken to prevent secondary infections from occurring and animals are tested for C. difficile carriage by culturing the bacterium from faeces. One day prior to infection (e.g., day −1), animals are pre-treated with clindamycin-HCl (10 mg/kg) to predispose them to C. difficile infection. Animals are then split into one of four groups. Beginning one day prior to C. difficile infection (day −1) to four days post infection (day 4), animals (6 per group) are treated with Lactobacillus by feeding animals 10⁴ to 10¹⁰ (e.g., 10¹⁰, 100 μL) CFU of Lactobacillus, each day. One group is fed a Lactobacillus control strain that does not express any scFv, one group is fed a Lactobacillus strain that expresses surface bound 6cdtA scFv (anti-Toxin A), one group is fed a Lactobacillus strain expressing surface bound 10cdtB scFv (anti-Toxin B), one group is fed a mixture of Lactobacillus strains expressing surface bound 6cdtA scFv (anti-Toxin A) and surface bound 10cdtB scFv (anti-Toxin B). A control group is not fed any Lactobacilli. On day 0, animals are infected with C. difficile spores (e.g., 20 spores, at least 200 times the LD100) by oral gavage.

From the day after infection (e.g., day 1), animals are observed several times per day for general physical appearance, signs of diarrhea (number and nature of faeces) and survival. Body weights and daily food intake are also monitored. Animals in extreme distress are sacrificed. The day of sacrifice or last day alive is considered the survival endpoint. Faeces are also collected for culturing to confirm C. difficile infection and evaluate Lactobacillus colonization.

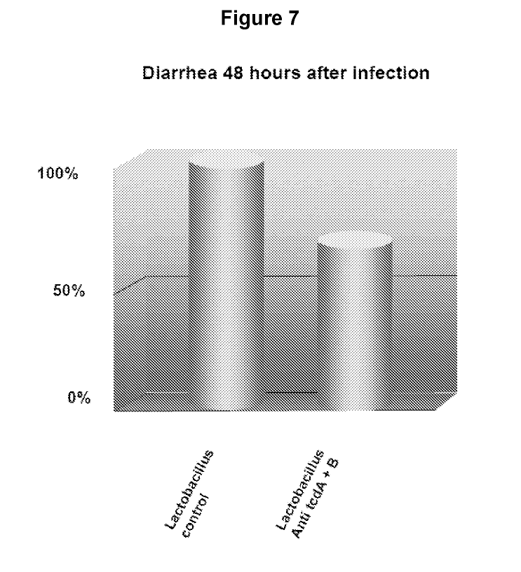

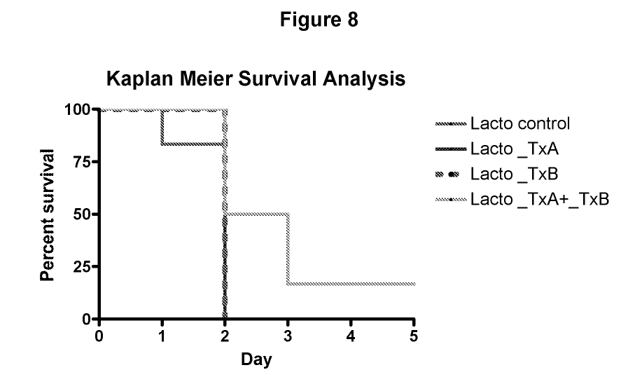

Animals showed no signs of distress or discomfort during and immediately after infection and treatment by oral gavage. Protection from diarrhea as shown in FIG. 7 and from death as shown in FIG. 8 was observed in the group treated with anti-Toxin A and anti-Toxin B. The survival advantage in the group treated with anti-Toxin A and anti-Toxin B was statistically significant (p=0.043) relative to the other treated groups.

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for treating a gastrointestinal disease in a subject in need thereof, said method comprising: administering to the subject a recombinant bacterium comprising one or more binding peptides, antibodies or fragments thereof anchored to its surface and specific for one or more pathogens or toxins.

2. The method of claim 1, wherein the subject has mild diarrhea, fatal pseudomembranous colitis or C. difficile associated diarrhea (CDAD).

3. The method of claim 1, wherein the bacterium is a Lactobacillus strain selected from the group consisting of: L. casei, L. paracasei, L. zeae, L. reuteri, L. plantarum, L. acidophilus, L. gasseri, L. brevis and combinations thereof.

4. (canceled)

5. The method of claim 1, wherein the pathogen is a bacterium native to the gastrointestinal tract selected from the group consisting of: enterotoxicogenic Escherichia coli, Campylobacter jejuni, Cryptosporidium spp., Giardia lamblia, Yersinia enterocolitica, Helicobacter pylori, all Clostridium spp., Vibrio cholera, C. difficile and combinations thereof.

6. (canceled)

7. (canceled)

8. The method of claim 1, wherein the pathogen is selected from the group consisting of Salmonella, Shigella Listeria spp. and combinations thereof.

9. (canceled)

10. The method of claim 1, wherein the pathogen is a virus.

11. The method of claim 10, wherein the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

12. (canceled)

13. (canceled)

14. (canceled)

15. The method of claim 1, wherein the antibody binding fragment is selected from the group consisting of: Fab, Fab′, Fab′-SH, Fv, scFv, F(ab′)₂, Vhh, nanobody and diabody.

16. The method of claim 1, wherein the antibody or fragments thereof are specific for Toxin A, Toxin B or surface antigens on C. difficile cells.

17. A recombinant bacterium comprising one or more binding peptides, antibodies or binding fragments thereof anchored to its surface and specific for one or more pathogens.

18. The recombinant bacterium of claim 17, wherein the bacterium is a Lactobacillu strain selected from the group consisting of: L. casei, L. paracasei, L. zeae, L. reuteri, L. plantarum, L. acidophilus, L. gasseri L. brevis and combinations thereof.

19. (canceled)

20. The pathogen of claim 17, wherein the pathogen is a bacterium native to the gastrointestinal tract selected from the group consisting of: enterotoxicogenic Escherichia coli, Campylobacter jejuni, Cryptosporidium spp., Giardia lamblia, Yersinia enterocolitica, Helicobacter pylori, all Clostridium spp., Vibrio cholera, C. difficile and combinations thereof.

21. (canceled)

22. (canceled)

23. The pathogen of claim 17, wherein the pathogen is selected from the group consisting of Salmonella, Shigella Listeria spp. and combinations thereof.

24. (canceled)

25. The pathogen of claim 17, wherein the pathogen is a virus.

26. The virus of claim 25, wherein the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

27. (canceled)

28. (canceled)

29. (canceled)

30. The antibody binding fragment of claim 17, wherein the antibody binding fragment is selected from the group consisting of: Fab, Fab′, Fab′-SH, Fv, scFv, F(ab′)₂, Vhh, nanobody and diabody.

31. The antibody or antibody binding fragment of claim 29, wherein the antibody or fragments thereof are specific for Toxin A, Toxin B or surface antigens on C. difficile cells.

32. A composition for treating a gastrointestinal disease, said composition comprising: a recombinant bacterium comprising one or more binding peptides, antibodies or fragments thereof anchored to its surface and specific for one or more pathogens.

33. The recombinant bacterium of claim 32, wherein the bacterium is a Lactobacillus strain selected from the group consisting of: L. casei, L. paracasei, L. zeae, L. reuteri, L plantarum, L. acidophilus, L. gasseri L. brevis and combinations thereof.

34. (canceled)

35. The pathogen of claim 32, wherein the pathogen is a bacterium native to the gastrointestinal tract selected from the group consisting of: enterotoxicogenic Escherichia coli, Campylobacter jejuni, Cryptosporidium spp., Giardia lamblia, Yersinia enterocolitica, Helicobacter pylori, all Clostridium spp., C. difficile, Vibrio cholera and combinations thereof.

36. (canceled)

37. (canceled)

38. The pathogen of claim 32, wherein the pathogen is selected from the group consisting of Salmonella, Shigella Listeria sp and combinations thereof.

39. (canceled)

40. The pathogen of claim 32, wherein the pathogen is a virus.

41. The virus of claim 40, wherein the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

42. (canceled)

43. (canceled)

44. (canceled)

45. The antibody binding fragment of claim 32, wherein the antibody binding fragment is selected from the group consisting of: Fab, Fab′, Fab′-SH, Fv, scFv, F(ab′)₂, Vhh, nanobody and a diabody.

46. The antibody or antibody binding fragment of claim 32, wherein the antibody or fragments thereof are specific for Toxin A, Toxin B or surface antigens on C. difficile cells.

47. A method for binding a pathogen or toxin in a gastrointestinal tract, said method comprising: administering to a subject in need thereof a recombinant bacterium that comprises one or more binding peptides, antibodies or binding fragments thereof anchored to its surface which are specific for one or more pathogens or toxins.

48. The method of claim 47, wherein the bacterium is a Lactobacillus strain selected from the group consisting of: L. casei, L. paracasei, L. zeae, L. reuteri, L. plantarum, L. acidophilus, L. gasseri, L. brevis and combinations thereof.

49. (canceled)

50. The method of claim 47, wherein the pathogen is a bacterium native to the gastrointestinal tract selected from the group consisting of: enterotoxicogenic Escherichia coli, Campylobacter jejuni, Cryptosporidium spp., Giardia lamblia, Yersinia enterocolitica, Helicobacter pylori, all Clostridium spp, C. difficile, Vibrio cholera and combinations thereof.

51. (canceled)

52. (canceled)

53. The method of claim 47, wherein the pathogen is selected from the group consisting of Salmonella, Shigella Listeria sp and combinations thereof.

54. (canceled)

55. The method of claim 47, wherein the pathogen is a virus.

56. The method of claim 55, wherein the virus is selected from the group consisting of: rotavirus, enteroviruses, adenoviruses, caliciviruses, reoviruses, coronaviruses, Norwalk-type viruses, coxsackieviruses, poliovirus and hepatitis A virus.

57. (canceled)

58. (canceled)

59. (canceled)

60. The method of claim 47, wherein the antibody binding fragment is selected from the group consisting of: Fab, Fab′, Fab′-SH, Fv, scFv, F(ab′)₂, Vhh, nanobody and diabody.

61. The method of claim 47, wherein the antibody or fragments thereof are specific for Toxin A, Toxin B or surface antigens on C. difficile cells.

62. (canceled)

63. The method of claim 1, wherein another agent is co-administered with the bacterium.

64. (canceled)

65. (canceled)

66. The antibody or antibody binding fragment of claim 30, wherein the antibody or fragments thereof are specific for Toxin A, Toxin B or surface antigens on C. difficile cells.

67. The method of claim 47, wherein another agent is co-administered with the bacterium.
